(19) United States
(12) Patent Application Publication
(10) Pub. No.: US 2021/0338575 A1
(43) Pub. Date: Nov. 4, 2021

(54) DRY PHARMACEUTICAL COMPOSITION FOR INHALATION

(71) Applicant: Deutsches Krebsforschungszentrum, Heidelberg (DE)

(72) Inventors: Martin Mueller, Heidelberg (DE); Irene Rossi, Heidelberg (DE); Gloria Spagnoli, Heidelberg (DE); Angelo Bolchi, Heidelberg (DE); Simone Ottonello, Heidelberg (DE); Ruggero Bettini, Heidelberg (DE)

(21) Appl. No.: 17/286,293

(22) PCT Filed: Oct. 17, 2019
(86) PCT No.: PCT/EP2019/078277

§ 371 Date: Apr. 16, 2021

(30) Foreign Application Priority Data
Oct. 18, 2018 (EP) 18201253.4

(51) Int. Cl. A61K 9/00; A61K 47/14; A61K 39/12

(52) U.S. Cl. CPC A61K 9/0075; A61K 2039/544; A61K 39/12; A61K 47/14

(57) ABSTRACT

The present invention relates to a dry pharmaceutical composition for inhalation comprising an antigen and an amphiphilic immune stimulant, wherein said pharmaceutical composition was produced by spray-drying; and to a dry pharmaceutical composition obtained or obtainable by spray-drying a solution comprising an antigen, an amphiphilic immune stimulant, and, optionally, a bulking agent. The present invention also relates to said dry pharmaceutical composition for use in medicine, in particular for use in vaccination of a subject; and to methods and kits related thereto.

The present invention relates to a dry pharmaceutical composition for inhalation comprising an antigen and an amphiphilic immune stimulant, wherein said pharmaceutical composition was produced by spray-drying; and to a dry pharmaceutical composition obtained or obtainable by spray-drying a solution comprising an antigen, an amphiphilic immune stimulant, and, optionally, a bulking agent. The present invention also relates to said dry pharmaceutical composition for use in medicine, in particular for use in vaccination of a subject; and to methods and kits related thereto.

Vaccination against infectious agents typically comprises subcutaneous or intramuscular injection of a solution or suspension containing an appropriate antigen. The usually poor physico-chemical stability of said solution/suspension under ambient temperature conditions (10-40° C. depending on the season and the specific geographic area) requires storage, transport and distribution of the vaccine product under temperature-controlled conditions (so called ‘cold-chain’). This significantly increases vaccine cost and dramatically limits vaccine availability/applicability in under-developed settings, where vaccination is usually more needed due to the lack of other/alternative prevention strategies (e.g., early cytological diagnosis). Associated to this cost increase and reduced availability, there is also a concern that the vaccine, during storage/transportation, may be accidentally exposed to either freezing or over-temperature conditions, both of which may seriously affect its efficacy.

Also, vaccine administration by injection may be painful, it is often accepted with diffidence by the patients and may generate adverse psychosomatic effects (so called ‘lipothymic reaction’), and it has to be performed by specialized healthcare personnel. The latter point as well as the diffidence associated with certain ethnic/religious groups further complicates and interferes with large-scale vaccination programs in underdeveloped, but also in developed countries.

As an alternative, administration of liquid formulations through the use of nebulizers, which transform the liquid into an aerosol, have been proposed (e.g. Nardelli-Haefliger et al. (2005), Vaccine, 23:3634-3641). This mode of delivery is particularly useful for the administration of large doses of an active and fairly stable pharmaceutical ingredient. Even though it can also be applied to a stabilized powder, dissolved under semi-sterile conditions immediately before use, aerosol-based administration comes with multiple drawbacks that significantly limit its use and applicability, namely that it is time-consuming, it requires a source of electrical power, and a significant part of the nebulized dose is dispersed into the environment.

As a further alternative, the use of dry formulations for inhalation was proposed and has become a standard method of administration, e.g. in asthma treatment. To improve galenic properties of dry powder preparations, lipids were proposed for combination, e.g. with antibiotic or chemotherapeutic substances (cf. e.g. WO 2010/003465).

Nonetheless, there is a need in the art to provide improved means and methods for immunization. In particular, there is a need to provide means and methods avoiding at least in part the drawbacks of the prior art as discussed above.

This problem is solved by the subject matter of the present invention with the features of the independent claims. Preferred embodiments, which might be realized in an isolated fashion or in any arbitrary combination are listed in the dependent claims.

Accordingly, the present invention relates to a dry pharmaceutical composition for inhalation comprising an antigen and an amphiphilic immune stimulant, wherein said pharmaceutical composition was produced by spray-drying.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and arc not intended to restrict the scope of the claims in any way. The invention may, as the skilled person will recognize, be performed by using alternative features. Similarly, features introduced by “in an embodiment” or similar expressions arc intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “pharmaceutical composition”, as used herein, relates to a composition comprising at least the specified compounds, i.e. at least the antigen and the amphiphilic immune stimulant, both as specified elsewhere herein, in a pharmaceutically acceptable form and, optionally, in combination with at least one pharmaceutically acceptable carrier, in particular at least one bulking agent, as specified elsewhere herein. The compounds can be provided as pharmaceutically acceptable salts; acceptable salts comprise in particular acetates, hydrochlorides, sulfates, chlorides, and the like.

Suitable routes of administration conventionally used for drug administration arc topical or systemic administration, in particular arc oral, intravenous, or parenteral administration, as well as inhalation. The pharmaceutical composition, as referred to herein, is a pharmaceutical composition for inhalation. Thus, the pharmaceutical composition is suitable for administration via inhalation. Preferably, the pharmaceutical composition for inhalation is administered via inhalation. As used herein, the term “inhalation” relates to taking in air or another gaseous carrier into the body of a subject, said air or gaseous carrier comprising a pharmaceutical composition as specified herein. Thus, inhalation, preferably, causes contacting at least part of the respiratory system of a subject with the pharmaceutical composition. As is understood by the skilled person, inhalation may be inhalation via the mouth and/or inhalation via the nose. Thus, preferably, inhalation comprises contacting at least one of the oral cavity, the nasal cavity, the pharynx, the trachea, and the lung of a subject with the pharmaceutical composition. Thus, more preferably, inhalation causes contacting of the epithelia of the oral cavity, the nasal cavity, the pharynx, the trachea, and/or the lung with the pharmaceutical composition. Thus, preferably, the pharmaceutical composition is administered topically to the aforesaid body parts.

The pharmaceutical composition is a dry pharmaceutical composition; thus, preferably, the pharmaceutical composition is a powder, preferably a dry powder; more preferably, the pharmaceutical composition has a water content of at most 5%, more preferably at most 2%, most preferably at most 1%. Preferably, the dry pharmaceutical composition comprises particles with a median volume diameter of at most 10 μm, more preferably at most 7.5 μm, most preferably at most 5 μm. Also preferably, the dry pharmaceutical composition comprises particles with a median volume diameter of from 0.5 μm to 10 μm, preferably of from 1 μm to 7.5 μm, more preferably of from 1.5 μm to 5 μm.

The pharmaceutical composition is produced by spray-drying, i.e. is produced by a process comprising a spray-drying step. The term “spray-drying” is, in principle, known to the skilled person. As used herein, the term preferably relates to a process step comprising spraying a solution comprising the compounds as specified at elevated temperature into a stream of a gaseous drying agent, e.g. heated air. Preferred embodiments of the drying step are specified elsewhere herein, in particular in the context of the method for manufacturing a dry pharmaceutical composition and in the Examples. Thus, the pharmaceutical composition preferably is obtained or obtainable by spray-drying a solution comprising an antigen, an amphiphilic immune stimulant, and, optionally, a bulking agent, preferably according to the method for manufacturing a dry pharmaceutical composition according to the present invention as specified herein below. In addition, the pharmaceutical composition may also include other carriers, adjuvants, or nontoxic, nontherapeutic, nonimmunogenic stabilizers and the like.

Preferably, the antigen is comprised in the dry pharmaceutical composition at a proportion of from 0.1% (w/w) to 10% (w/w), preferably of from 0.2% (w/w) to 5% (w/w), more preferably of from 0.3% (w/w) to 2.5% (w/w), most preferably of about 2% (w/w). Also preferably, the amphiphilic immune stimulant is comprised in the dry pharmaceutical composition at a proportion of from 0.05% (w/w) to 10% (w/w), preferably of from 0.1% (w/w) to 5% (w/w), more preferably of from 0.15% (w/w) to 2% (w/w), most preferably of about 0.2% (w/w). Also preferably, the ratio of bulking agent:antigen is of from 1000:1 (w/w) to 10:1 (w/w), preferably of from 500:1 (w/w) to 25:1 (w/w), more preferably of from 300:1 (w/w) to 30:1 (w/w), even more preferably is about 49:1 (w/w), most preferably is 49 (w/w). Preferably, the amphiphilic immune stimulant at least partially coats the antigen in the dry pharmaceutical composition. Preferably, the dry pharmaceutical composition comprises particles consisting of a core portion and coating portion, wherein said core portion comprises most of the antigen and, optionally the bulking agent, and wherein said coating portion comprises most of the amphiphilic immune stimulant. As will be understood by the skilled person, the term “comprises most of”, as used herein, relates to the fact that a portion, preferably a substructure, of the particles of the dry pharmaceutical composition comprises the predominant portion of the total amount of the indicated compound. Thus, preferably, the dry pharmaceutical composition comprises particles consisting of a core portion comprising at least 75%, preferably at least 85%, more preferably at least 90% of the total amount of antigen and, optionally at least one bulking agent, comprised in said particles; and of a coating portion comprising at least 75%, preferably at least 85%, more preferably at least 90% of the total amount of amphiphilic immune stimulant comprised in said particles. Thus, preferably, the dry pharmaceutical composition consists of particles consisting essentially of antigen and, if present, bulking agent, coated by an amphiphilic immune stimulant. Moreover, the pharmaceutical composition may comprise further drugs in a common pharmaceutical composition; also, the pharmaceutical compositions may be used in combination with further pharmaceutical compositions, which may be provided in form of a kit of parts.

A therapeutically effective dose refers to an amount of the compounds to be used in a dose of the pharmaceutical composition which prevents, ameliorates or treats a condition referred to herein. Therapeutic efficacy and toxicity of compounds can be determined by standard pharmaceutical procedures in cell culture or in experimental animals, e.g., by determining the ED₅₀ (the dose therapeutically effective in 50% of the population) and/or the LD₅₀ (the dose lethal to 50% of the population). The dose ratio between therapeutic and toxic effects is the therapeutic index, and it can be expressed as the ratio, LD₅₀/ED₅₀. The dosage regimen will be determined by the attending physician, preferably taking into account relevant clinical factors and, preferably, in accordance with any one of the methods described elsewhere herein. As is well known in the medical arts, a dosage for any one patient may depend upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 μg to 10000 μg; however, doses below or above this exemplary range are envisioned, especially considering the aforementioned factors. Generally, the regimen comprises administration of 1 μg to 10 mg of an antigen as a primary immunization, followed by one or more than one boost administration of the same antigen, preferably in the same dosage. However, depending on the subject and the mode of administration, the quantity of substance administration may vary over a wide range to provide from about 0.01 mg per kg body mass to about 1 mg per kg body mass, preferably. The pharmaceutical compositions and formulations referred to herein arc administered at least once in order to treat or prevent a disease or condition recited in this specification. However, the said pharmaceutical compositions may be administered more than one time, for example, preferably from one to four times, more preferably two or three times.

Preferably, the pharmaceutical composition further comprises at least one pharmaceutically acceptable carrier (excipient), in particular a bulking agent. It will be appreciated that the form and character of the pharmaceutically acceptable carrier or diluent is dictated by the amount of active ingredient with which it is to be combined, the route of administration and other well-known variables. The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers arc sugars, sugar alcohols, terra alba, talc, gelatin, agar, pectin, acacia, magnesium stearate, stearic acid and the like. Similarly, the carrier or diluent may include time delay material well known to the art, such as glyceryl mono-stearate or glyceryl distearate alone or with a wax. More preferably, the excipient comprises, preferably is, at least one bulking agent. The term “bulking agent”, as used herein, relates to a pharmaceutically acceptable excipient included in the pharmaceutical composition to increase the overall mass of the product, in particular to increase the mass and/or size of the particles of the pharmaceutical composition. Thus, preferred bulking agents arc pharmaceutically inert compounds, which are preferably non-hygroscopic and, also preferably, crystallize well. Thus, preferred bulking compounds arc pharmaceutically inert sugar or sugar alcohol compounds, preferably selected from mannitol, lactose, and trehalose. Preferably, the bulking agent comprises, more preferably is, mannitol.

The term “antigen” is understood by the skilled person to relate to any chemical compound or mixture thereof capable of inducing an immune response in a subject, preferably under appropriate conditions. Preferably, the antigen is a biological polymer, more preferably is a biological macromolecule, still more preferably is a biological macromolecule having a molecular mass of at least 1000 Da, more preferably at least 5000 Da, most preferably is a polypeptide. Preferably, the antigen comprises a thioredoxin or a fragment thereof, more preferably a thioredoxin of a thermophilic organism, preferably a thermophilic archaeon, more preferably of Pyrococcus furiosus, or a fragment thereof. Still more preferably, the antigen comprises a thioredoxin, more preferably a thioredoxin of a thermophilic organism, preferably a thermophilic archaeon, more preferably of Pyrococcus furiosus. Preferably, the aforesaid thioredoxin has the function of a scaffold, i.e. as a compound providing a stabilizing framework for at least one antigenic epitope, the term “antigenic epitope” referring to a structure of a biological macromolecule, preferably a polypeptide, being presented in the body of a subject to the immune system, preferably via major histocompatibility molecules (MHCs) and/or being a binding site for antibodies. Thus, preferably, the antigenic epitope is an antigenic fragment of a polypeptide, i.e. an antigenic peptide. Preferably, the antigenic epitope is antigenic epitope of a papillomavirus, preferably comprising at least one peptide comprising an amino acid sequence corresponding to amino acids 20 to 38 of the HPV16 L2 polypeptide, more preferably comprising at least three peptides comprising an amino acid sequence corresponding to amino acids 20 to 38 of the HPV16 L2 polypeptide, still more preferably comprising at least three peptides comprising the amino acid sequence of amino acids 20 to 38 of the HPV16 L2 polypeptide, most preferably comprising three peptides consisting of the amino acid sequence of amino acids 20 to 38 of the HPV16 L2 polypeptide. Thus, preferred antigens arc the immunogenic polypeptides as specified in WO 2010/070052 and WO 2017/211886. Preferably, the antigen is thermostable, more preferably a thermostable antigen, wherein the term “thermostable” relates to the property of a compound of essentially maintaining its chemical properties, more preferably its immunogenic properties, after an incubation in an aqueous solution at pH=7 for at least 20 min at 50° C., more preferably at 60° C., even more preferably at 70° C. In case the antigen is a polypeptide, thermostability can preferably also be determined by determining circular dichroism of a solution of said polypeptide in the presence of ethanol; as used herein, a polypeptide is preferably deemed thermostable if the circular dichroism determined under standard conditions in the range of 200 nm to 260 nm is essentially unchanged in an aqueous solution comprising up to 20% ethanol, preferably up to 30% ethanol, more preferably up to 50% ethanol, compared to the same polypeptide in the same aqueous solution in the absence of ethanol.

The term “immune stimulant”, as used herein, relates to a chemical compound increasing the amplitude of an immune response of a subject, while, preferably, itself not being immunogenic. Thus, preferably, the immune stimulants are immunologic adjuvants, which arc in principle known to the skilled person. Preferably, the immune stimulant is an agonist of a toll-like receptor (TLR), preferably of TLR4. As related to herein, the immune stimulant is an “amphiphilic immune stimulant”; thus, the immune stimulant has lipophilic and hydrophilic properties. Also preferably, the amphiphilic immune stimulant has an overall extended structure with a hydrophilic end and a lipophilic end. Preferably, the amphiphilic immune stimulant forms an ordered monomolecular array or layer at an air-water interface. Also preferably, the amphiphilic immune stimulant forms micelles at an increased concentration in an aqueous solution; thus, preferably, the amphiphilic immune stimulant has a critical micelle concentration (CMC); more preferably, the amphiphilic immune stimulant has a CMC value of at most 1 mM, preferably at most 100 μM, more preferably at most 10 μM. Preferably, the amphiphilic immune stimulant is a force controlling agent, more preferably, the amphiphilic immune stimulant is a surfactant. Preferably, the amphiphilic immune stimulant is selected from the list consisting of monophosphoryl lipid A, synthetic lipid A, lipid A analogs, lipid A mimetics, cytokines, saponins, lipopolysaccharide (LPS) of gram-negative bacteria, and endotoxins. More preferably, the amphiphilic immune stimulant is monophosphoryl lipid A (CAS NO: 143110-73-0)

Advantageously, it was found in the work underlying the present invention that the dry pharmaceutical compositions of the present invention allow for a highly efficient, non-invasive immunization. Also, the amphiphilic immune stimulant acts as both a biologically active component potentiating the immune response, and as a technological excipient that improves powder flowability, aerosolization performance and respirability, while enhancing antigen stability and reducing sensitivity to environmental humidity. Moreover, the dry pharmaceutical compositions were found to be stable even at ambient temperature and, thus, arc particularly suitable for use in regions where cooling of vaccines cannot be ensured.

The definitions made above apply mutatis mutandis to the following. Additional definitions and explanations made further below also apply for all embodiments described in this specification mutatis mutandis.

The present invention further relates to a dry pharmaceutical composition according to present invention for use in medicine; the present invention also relates to a dry pharmaceutical composition according to present invention for use in treating and/or preventing an infection in a subject, preferably, for use in vaccination of a subject.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values arc, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population.

The term “preventing” refers to retaining health with respect to the diseases or disorders referred to herein for a certain period of time in a subject. It will be understood that the said period of time may be dependent on the amount of the drug compound which has been administered and individual factors of the subject. It is to be understood that prevention may not be effective in all subjects treated with the compound according to the present invention. However, the term requires that, preferably, a statistically significant portion of subjects of a cohort or population arc effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

The term “vaccination” is, in principle, known to the skilled person to relate to an administration of at least one antigenic structure, in particular at least one epitope, to a subject to modulate an immune response to compounds comprising said antigenic structure. Preferably, said modulation is inhibition of an immune response; more preferably, said modulation is activation of an immune response. The skilled person will understand that vaccination may not elicit a significant immune response in all subjects vaccinated. Also, it is to be understood that vaccination may not be effective to prevent infection in all subjects vaccinated. However, the term requires that a, preferably statistically significant, portion of subjects of a cohort or population arc effectively vaccinated. In case the infectious agent is a HPV, effective vaccination, preferably, is prevention or reduction of the number or HPV-induced lesions, such as warts.

Preferably, vaccination is therapeutic vaccination, i.e. vaccination to treat a disease or disorder entailing the presence of the antigenic structure in the body of a subject. Preferably, therapeutic vaccination induces tolerance, preferably peripheral tolerance, to the antigenic structure used for vaccination, e.g. in allergic or autoimmune diseases; methods of inducing tolerance are known in the art and include in particular repeated administration of an (auto)antigen, inhibition of CD28 signaling in T cells, and stimulation of regulatory T cells. More preferably, therapeutic vaccination elicits or stimulates immunity, i.e. a defense reaction against the antigenic structure used for vaccination, e.g. in cancer or in pre-existing persistent infections, e.g. latent viral infections. Thus, preferred antigenic structures are derived from tumor antigens, tumor specific antigens, and/or genes expressed by infectious agents during persistent infection, e.g. latent genes of viruses.

More preferably, vaccination is preventive vaccination, i.e. vaccination to prevent a disease or disorder entailing the presence of the antigenic structure used for vaccination in the body of a subject. Thus, preferably, in case of disease caused by an infectious agent, preventing as specified above may be prophylactic vaccination. Thus, preferably, the term preventing relates to administering the compounds as specified herein to elicit an immune response against at least one infectious agent. Thus, preferably, vaccination stimulates the immune system and establishes or improves immunity to infection with infectious agents. Preferably, vaccination according to the present invention allows for establishing or improving immunity to infection with an infectious agent, preferably human papillomavirus genotypes. It is to be understood that the vaccine according to the present invention may comprise further components, in particular as specified elsewhere herein.

The term “infectious agent”, as used herein, relates to an agent, preferably a microorganism, causing transmissible disease in a subject. Preferably, the infectious agent is a bacterium, an eukaryotic infectious agent, e.g. a Plasmodium spp., or a virus, more preferably is a virus, e.g. a Papillomavirus, a Hepatitis virus or Human Immunodeficiency Virus (HIV). More preferably, the infectious agent is an oncogenic virus, more preferably a Papillomavirus, an Epstein-Barr virus, a Hepatitis virus, a Human T-lymphotropic virus 1, a Human herpesvirus 8, more preferably a Papillomavirus (PV), most preferably a human Papillomavirus (HPV). Preferably, the infectious agent is an agent causing chronic disease. More preferably, the infectious agent is an agent causing chronic and/or persisting infection.

The term “subject”, as used herein, relates to an animal, preferably a vertebrate, more preferably a mammal, in particular to livestock like cattle, horse, pig, sheep, and goat, or to a laboratory animal like a rat, mouse, and guinea pig. Most preferably, the subject is a human.

The present invention further relates to a method for manufacturing a dry pharmaceutical composition comprising spray-drying a solution comprising an antigen, an amphiphilic immune stimulant, and, optionally, a bulking agent.

The method of the present invention is an in vitro method. Moreover, it may comprise steps in addition to those explicitly mentioned above. For example, further steps may relate, e.g., to providing a solution comprising the indicated compounds for spray-drying, and/or further manufacturing steps relating to the spray-dried product. Moreover, one or more of said steps may be performed by automated equipment. Preferably, the method further comprises admixing the amphiphilic immune stimulant to a solution comprising said antigen and, optionally, said bulking agent. Also preferably, the aforesaid solution further comprises an ethanol/water mixture as a solvent. Preferably, said solvent comprises of from 10% (v/v) to 50% (v/v) ethanol in water, more preferably of from 20% (v/v) to 40% (v/v), even more preferably about 30% (v/v), most preferably 30% (v/v). Thus, preferably, the ethanol/water ratio in said solvent is of from 2:1 (v/v) to 10:1 (v/v), preferably is of from 5:1 (v/v) to 9:1 (v/v), more preferably of from 6:1 (v/v) to 8:1 (v/v), most preferably is about 70% (v/v).

Preferably, the solution comprising the antigen and the amphiphilic immune stimulant comprises the antigen at a concentration of from 0.01 mg/ml to 1 mg/ml, preferably of from 0.025 mg/ml to 0.75 mg/ml, more preferably of from 0.05 mg/ml to 0.5 mg/ml, even more preferably of from 0.075 mg/ml to 0.25 mg/ml, still more preferably of about 0.1 mg/ml, most preferably of 0.1 mg/ml. Also preferably, the solution comprising the antigen and the amphiphilic immune stimulant comprises the amphiphilic immune stimulant at a concentration of from 0.1 μg/ml to 1 mg/ml, preferably of from 1 μg/ml to 500 μg/ml, more preferably of from 10 μg/ml to 250 μg/ml, still more preferably of about 15 μg/ml, most preferably of 10.4 μg/ml. Also preferably, the concentration of the bulking agent, preferable mannitol, in the solution further comprising the antigen and the amphiphilic immune stimulant is of from 0.1 mg/ml to 3 mg/ml, preferably of from 0.25 mg/ml to 1.5 mg/ml, more preferably of from 0.4 mg/ml to 1 mg/ml, even more preferably of from 0.5 mg/ml to 0.75 mg/ml, still more preferably of about 0.6 mg/ml, most preferably of 0.59 mg/ml.

As indicated herein above, the term “spray-drying” is understood by the skilled person and preferably relates to a process step comprising spraying a solution comprising the compounds as specified at elevated temperature into a stream of a gaseous drying agent, e.g. heated air. Preferably, spray-drying comprises heating the drying gas stream, preferably the drying air stream, to a temperature of from 80° C. to 150° C., preferably of from 100° C. to 140° C., most preferably about 125° C. Preferably, the gas flow is of from 100 l/h to 1000 l/h, more preferably of from 250 l/h to 750 l/h, still more preferably 500 l/h to 700 l/h, most preferably about 600 l/h, preferably with an aspiration of from 10 m³/h to 100 m³/h, more preferably of from 15 m³/h to 65 m³/h, still more preferably of from 25 m³/h to 50 m³/h, most preferably about 37 m³/h. Preferably, the particle size distribution of the dry pharmaceutical composition is Dv10 1.43+/−0.09 μm; Dv50 2.65+/−0.17 μm; and/or Dv90 4.78+/−0.55 μm.

The present invention also relates to a kit comprising the dry pharmaceutical composition according to the present invention in a housing.

The term “kit”, as used herein, refers to a collection of the aforementioned compounds, means or reagents which may or may not be packaged together. Preferably, the kit is used for practicing the medical uses of the pharmaceutical composition referred to herein above. It is, preferably, envisaged that all components are provided in a ready-to-use manner for practicing the uses referred to above. Further, the kit, preferably, contains instructions for carrying out said uses. The instructions can be provided by a user's manual in paper or electronic form. In addition, the manual may comprise instructions for administration and/or dosage instructions for carrying out the aforementioned uses using the kit of the present invention. Preferably, the housing of the kit comprises a dispensing means; thus, preferably, the housing is an inhaler, preferably an autoinhaler. Preferably, the kit comprises further components, e.g. an inhalation aid and/or an adapter. In such case, the components of the kit may be comprised by separate enclosures (i.e. as a kit of separate parts) or provides in an enclosure.

The present invention further relates to a use of a dry pharmaceutical composition according to the present invention for the manufacture of a medicament, preferably for use in the manufacture of a vaccine.

The present invention also relates to a method of treating and/or preventing an infection in a subject, comprising contacting said subject with a dry pharmaceutical composition according to any the present invention, thereby treating and/or preventing an infection.

The method of treating and/or preventing an infection of the present invention is an in vivo method. Moreover, it may comprise steps in addition to those explicitly mentioned above. For example, further steps may relate, e.g., to diagnosing an infection or a risk of acquiring an infection before step a), and/or providing additional therapeutic measures for treating preventing an infection and/or one of its comorbidities. Moreover, one or more of said steps may be performed by automated equipment.

In view of the above, the following embodiments are preferred:

1. A dry pharmaceutical composition for inhalation comprising an antigen and an amphiphilic immune stimulant, wherein said pharmaceutical composition was produced by spray-drying.
2. The dry pharmaceutical composition of claim 1 further comprising at least one bulking agent.
3. The dry pharmaceutical composition of claim 2, wherein said bulking agent is a sugar or sugar alcohol, preferably is selected from mannitol, lactose, and trehalose, more preferably is mannitol.
4. The dry pharmaceutical composition of any one of claims 1 to 3, wherein said dry pharmaceutical composition comprises particles consisting of a core portion comprising at least 75%, preferably at least 85%, more preferably at least 90% of the total amount of antigen and, optionally at least one bulking agent, comprised in said particles; and of a coating portion comprising at least 75%, preferably at least 85%, more preferably at least 90% of the total amount of amphiphilic immune stimulant comprised in said particles.
5. The dry pharmaceutical composition of any one of claims 1 to 4, wherein said dry pharmaceutical composition comprises particles with a median volume diameter of at most 10 μm, more preferably at most 7.5 μm, most preferably at most 5 μm.
6. The dry pharmaceutical composition of any one of claims 1 to 5, wherein said dry pharmaceutical composition comprises particles with a median volume diameter of from 0.5 μm to 10 μm, preferably of from 1 μm to 7.5 μm, more preferably of from 1.5 μm to 5 μm.
7. The dry pharmaceutical composition of any one of claims 1 to 6, wherein said antigen is comprised at a proportion of from 0.1% (w/w) to 10% (w/w), preferably of from 0.2% (w/w) to 5% (w/w), more preferably of from 0.3% (w/w) to 2.5% (w/w), most preferably of about 2% (w/w) in said dry pharmaceutical composition.
8. The dry pharmaceutical composition of any one of claims 1 to 7, herein said amphiphilic immune stimulant is comprised at a proportion of from 0.1% (w/w) to 10% (w/w), preferably of from 1% (w/w) to 5% (w/w), more preferably of from 0.5% (w/w) to 2% (w/w), most preferably of about 0.2% (w/w) in said dry pharmaceutical composition.
9. The dry pharmaceutical composition of any one of claims 2 to 8, wherein the ratio of bulking agent:antigen is of from 1000:1 (w/w) to 10:1 (w/w), preferably of from 500:1 (w/w) to 25:1 (w/w), more preferably of from 300:1 (w/w) to 30:1 (w/w), most preferably is about 49:1 (w/w).
10. The dry pharmaceutical composition of any one of claims 4 to 9, wherein said amphiphilic immune stimulant in said coating portion at least partially coats said core portion of said particles.
11. The dry pharmaceutical composition of any one of claims 1 to 10, wherein said amphiphilic immune stimulant is an agonist of a toll-like receptor (TLR), preferably of TLR4.
12. The dry pharmaceutical composition of any one of claims 1 to 11, wherein said amphiphilic immune stimulant is a force controlling agent.
13. The dry pharmaceutical composition of any one of claims 1 to 12, wherein said amphiphilic immune stimulant is selected from the list consisting of monophosphoryl lipid A, synthetic lipid A, lipid A analogs, lipid A mimetics, cytokines, saponins, lipopolysaccharide (LPS) of gram-negative bacteria, and endotoxins.
14. The dry pharmaceutical composition of any one of claims 1 to 13, wherein said amphiphilic immune stimulant is monophosphoryl lipid A.
15. The dry pharmaceutical composition of any one of claims 1 to 14, wherein said antigen is a biological macromolecule.
16. The dry pharmaceutical composition of any one of claims 1 to 15, wherein said antigen is a polypeptide.
17. The dry pharmaceutical composition of any one of claims 1 to 16, wherein said antigen is thermostable.
18. The dry pharmaceutical composition of any one of claims 1 to 17, wherein said antigen is a thermostable polypeptide.
19. The dry pharmaceutical composition of any one of claims 1 to 18, wherein said antigen comprises a thioredoxin.
20. The dry pharmaceutical composition of any one of claims 1 to 19, wherein said antigen comprises a thioredoxin of a thermophilic organism, preferably a thermophilic archaeon, more preferably of Pyrococcus furiosus.
21. The dry pharmaceutical composition of any one of claims 1 to 20, wherein said antigen comprises at least one antigenic epitope of a papillomavirus.
22. The dry pharmaceutical composition of any one of claims 1 to 21, wherein said antigen comprises at least one peptide comprising an amino acid sequence corresponding to amino acids 20 to 38 of the HPV16 L2 polypeptide.
23. The dry pharmaceutical composition of any one of claims 1 to 22, wherein said antigen comprises at least three peptides comprising an amino acid sequence corresponding to amino acids 20 to 38 of the HPV16 L2 polypeptide.
24. The dry pharmaceutical composition of any one of claims 1 to 23, wherein said pharmaceutical composition has a water content of at most 5%, more preferably at most 2%, most preferably at most 1%.
25. A dry pharmaceutical composition obtained or obtainable by spray-drying a solution comprising an antigen, an amphiphilic immune stimulant, and, optionally, a bulking agent, preferably according to the method according to any one of claims 31 to 35.
26. A dry pharmaceutical composition according to any one of claims 1 to 25 for use in medicine.
27. A dry pharmaceutical composition according to any one of claims 1 to 25 for use in vaccination of a subject.
28. The dry pharmaceutical composition for use of claim 27, wherein said antigen comprises at least one antigenic epitope of a papillomavirus and wherein said vaccination is vaccination against papillomavirus.
29. The dry pharmaceutical composition for use of any one of claims 26 to 28, wherein said use comprises administration by inhalation.
30. The dry pharmaceutical composition for use of any one of claims 25 to 29, wherein said subject is a mammal, preferably a human.
31. A method for manufacturing a dry pharmaceutical composition comprising spray-drying a solution comprising an antigen, an amphiphilic immune stimulant, and, optionally, a bulking agent.
32. The method of claim 31, wherein said method comprises admixing said amphiphilic immune stimulant to a solution comprising said antigen and, optionally, said bulking agent.
33. The method of claim 31 or 32, wherein said solution further comprises an ethanol/water mixture as a solvent.
34. The method of any one of claims 31 to 33, wherein the ethanol/water ratio in said solvent is of from 2:1 (v/v) to 10:1 (v/v), preferably is of from 5:1 (v/v) to 9:1 (v/v), more preferably of from 6:1 (v/v) to 8:1 (v/v), most preferably is about 70% (v/v).
35. The method of any one of claims 31 to 35, wherein said spray-drying comprises heating the drying air stream to a temperature of from 80° C. to 150° C., preferably of from 100° C. to 140° C., most preferably about 125° C.
36. A kit comprising the dry pharmaceutical composition according to any one of claims 1 to 30 in a housing.
37. The kit of claim 36, wherein said kit further comprises a means of administration or wherein said housing is a means of administration.
38. Use of a dry pharmaceutical composition according to any one of claims 1 to 30 for the manufacture of a pharmaceutical composition for use in the manufacture of a medicament, preferably for use in the manufacture of a vaccine.
39. A method of treating and/or preventing an infection in a subject, comprising contacting said subject with a dry pharmaceutical composition according to any one of claims 1 to 30, thereby treating and/or preventing an infection.

All references cited in this specification are herewith incorporated by reference with respect to their entire disclosure content and the disclosure content specifically mentioned in this specification.

### FIGURE LEGENDS

FIG. 1. SDS-PAGE fractionation and fluorescence analysis of soluble tissue supernatants (20,000×g, 15 min) derived from lung (L) and trachea (T) explants from mice injected intro-tracheally with the dry-powder formulated vaccine containing the PfTrx-HPV16-L2x3 antigen pre-labeled with Alexa Fluor 750. The results obtained with biological replicate tissue samples derived from two mice (L1, T1; L2, T2) arc shown; the Alexa Fluor 750-labeled input antigen (C+) and a lung tissue sample derived from a mouse not injected with the fluorescently labeled dry-powder vaccine (C−) served as positive and negative controls, respectively.

FIG. 2. Evaluation of the immunogenicity of the dry-powder formulated HPV-L2 vaccine. (A) Immunization and blood collection schedule. (B) Results obtained from GST-L2 ELISA testing of individual mice (represented by the indicated symbols) injected: i) subcutaneously with the antigen-lacking powder (#1; negative control); ii) subcutaneously with the soluble, doubly-adjuvanted vaccine (20 μg of PfTrx-HPV16-L2x3 antigen) (#2; positive control); intra-tracheally with the dry-powder formulated HPV-L2 vaccine (1 mg total powder corresponding to approximately 20 μg of the PfTrx-HPV16-L2x3 antigen) (#3; test sample); subcutaneously with the mono-adjuvanted, dry-powder pre-formulated vaccine (#4; same amount as in #3) dissolved in PBS. The measured anti-L2 antibody titers, determined with the use of a horseradish peroxidase-conjugated secondary antibody and the chromogenic substrate o-phenylenediamine (read spectrophotometrically at 450 nm), are shown on the y-axis. (C) Immunoglobulin isotypes determined at a fixed immune-serum dilution using rat anti-mouse Ig subclass-specific, horseradish peroxidase-conjugated secondary antibodies.

FIG. 3. Comparative analysis of immune sera derived from mice injected intra-tracheally with dry-powder formulated vaccines containing either the reference antigen PfTrx-HPV16-L2x3 (#3) or the modified OVX313-PfTrx-HPV-L2x3 antigen (#5) as active ingredients. Immunogenicity, expressed as anti-HPV-L2 antibody titers, was determined by GST-L2 capture ELISA as described in FIG. 2 legend.

The following Examples shall merely illustrate the invention. They shall not be construed, whatsoever, to limit the scope of the invention.

### EXAMPLE 1

The vaccine was produced by spray-drying, using mannitol as a bulking agent, starting from a 70:30 v/v water ethanol solution. The antigen was typically dissolved in a potassium phosphate solution at a concentration ranging from 1 to 25 mM. To prepare 100 mL of feed solution to be spray-dried, 58.7 mg of mannitol were dissolved in 69 mL of purified water, to which 1 mL of a potassium phosphate aqueous solution typically containing 10 mg of antigen was added. Monophosphoryl lipid A (MPLA; typically, 1.04 mg) was dissolved in 30 mL of ethanol (95% v/v) and the resulting solution was added to the pre-mixed solution containing mannitol and the antigen.

The final, complete solution was then sprayed with a Buchi 290 spray-drier set to the following process parameters: inlet temperature 125° C.; feed rate 3.5 ml/min; air flow 601 L/h; aspiration 37 m³/h.

The antigen concentration may range from 0.31 to 2.00% w/w with respect to the final formulation. When dissolved in water the powder yields a pH of 7.75. The particle size distribution of the resulting powder was Dv10 1.43+/−0.09 μm; Dv50 2.65+/−0.17 μm; Dv90 4.78+/−0.55 μm. The powder is stable at room temperature for at least 1 year.

The aerodynamic performance of the vaccine, measured upon in vitro/laboratory aerosolization according to Ph. Eur 0.9.0 with a RS01® (Plastiape) inhalation device, results in an emitted fraction of 81.35+/−11.2% of the loaded dose and a respirable fraction of 74.86+/−12.04% of the emitted dose.

### EXAMPLE 2

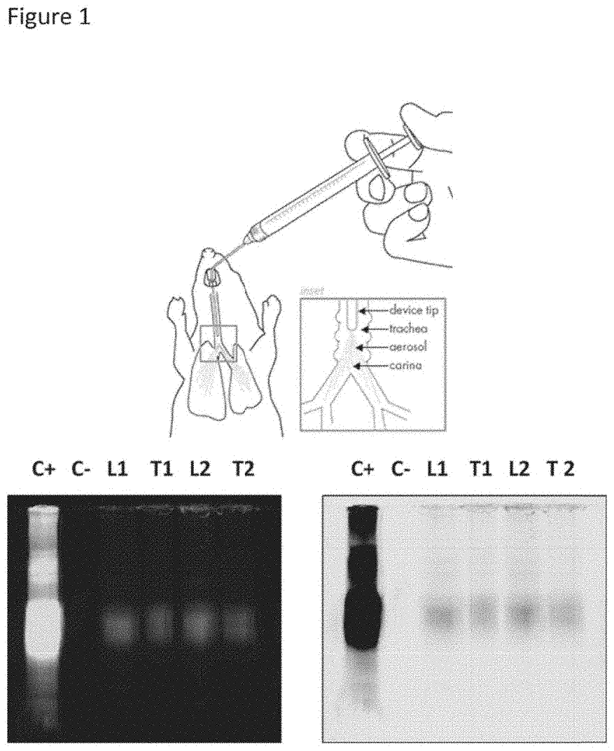

To monitor effective delivery of the dry-powder vaccine to the lungs, the antigenic protein (i.e., Pyrococcus furiosus thioredoxin displaying three tandem repeats of a HPV16-L2 peptide epitope spanning amino acid positions 20-38 of minor capsid protein L2; hereafter designated as PfTrx-HPV16-L2x3) was labeled with Alexa Fluor 750 prior to incorporation into the inhalator powder formulation by spray-drying. The vaccine-containing powder (1.7 and 2.0 mg corresponding to 10 μg and 12 μg of PfTrx-HPV16-L2x3 antigen, respectively) was then administered to two Balb/c mice with the use of a Penn Century microsprayer device. This was followed by mouse sacrifice and organ (lungs, trachea) explant after 15 min, tissue homogenization, centrifugation at 20,000×g for 15 min at 4° C., and fractionation of the resulting soluble supernatant on a denaturing, SDS-containing polyacrylamide gel (11%), which was then visualized by near-infrared fluorescence (NIR) imaging (Odyssey, Li-Cor). As shown in FIG. 1, a specific NIR signal associated to a polypeptide band displaying the molecular weight (18294.3 Da) expected for PfTrx-HPV16-L2x3 was detected in the lung (L) and in the trachea (T) samples.

### EXAMPLE 3

Having shown that the dry-powder formulated PfTrx-HPV16-L2x3 antigen can reach the respiratory tract upon mouse intra-tracheal delivery (a surrogate of autoinhaler-assisted self-administration in humans), the MPLA-adjuvanted dry-powder vaccine was evaluated for immunogenicity. To this end, the PfTrx-HPV16-L2x3 antigen was exchanged into PBS buffer and detoxified by Triton X-114 treatment, prior to spray-drying-mediated incorporation into the MPLA-containing powder and intra-tracheal administration with the use of a Penn Century microsprayer device to 6-8 weeks-old female BALB/c mice. These were subdivided into four groups:

1) A negative control group, consisting of 5 mice to which the empty powder (i.e., the MPLA-containing powder lacking the PfTrx-HPV16-L2x3 antigen as the ‘active ingredient’ (1 mg) was administered subcutaneously.

2) A positive control group consisting of 7 mice to which the soluble, Alum (50+MPLA (10 μg) adjuvanted PfTrx-HPV16-L2x3 vaccine (20 μg) was administered subcutaneously.

3) A test group consisting of 10 mice to which the dry-powder formulated PfTrx-HPV16-L2x3 vaccine (1 mg total powder corresponding to approximately 20 μg of protein antigen) was administered intra-tracheally.

4) An internal comparison group consisting of 7 mice to which the dry-powder pre-formulated vaccine (same amount as in #3) dissolved in PBS immediately prior to subcutaneous administration, was administered.

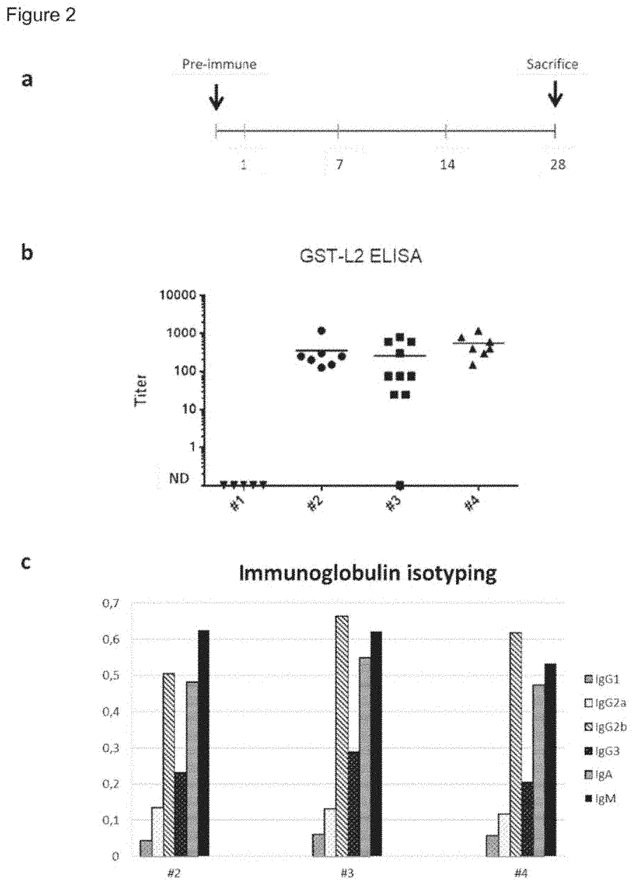

The immunization protocol consisted of a priming immunization, followed by two boosts at weekly intervals (FIG. 2a) and was preceded by the sampling of pre-immune scrum to be used as a background reference in subsequent immuno-assays. Two weeks after the last immunization, mice were sacrificed, blood was collected via cardiac puncture, and used for the preparation of immune-sera, which were stored at −80° C. till subsequent immunological analyses. These were conducted by an indirect GST-L2 capture enzyme-linked immunosorbent assay (ELISA) to measure total anti-L2 scrum antibody titers, and by a quantitative isotype-specific, indirect double sandwich ELISA to determine the specific immunoglobulin (Ig) isotypes and sub-types elicited by each type of immunization.

As shown in FIG. 2b, anti-HPV L2 antibody titers measured in group #3 (dry-powder vaccine delivered intra-tracheally) ranged from 25 to 800, with an average value of 1:255, which was not significantly different (P<0.2790) from the titer elicited by a standard subcutaneous immunization conducted with the doubly adjuvanted (Alum+MPLA) PfTrx-HPV16-L2x3 vaccine (group #2). Under the same experimental conditions, no above background immune response could be detected in the antigen-lacking negative control group (#1), whereas antibody titers similar to (or slightly higher than, but without statistical significance) those measured in the doubly-adjuvanted positive control group (#2) were detected upon subcutaneous immunization with the mono-adjuvanted, PBS-dissolved dry-powder vaccine.

As further shown in FIG. 2 (panel c), overall similar 1g isotypes (and sub-types) were measured in the three immunized groups (#2, #3 and #4), but with a reproducible skewing toward a prevalent IgG2b sub-type and a concomitant reduction of the early formed (and poorly matured) pentameric IgM isotype in the case of the dry-powder formulated vaccine, either administered directly (i.e., intra-tracheally; group #3) or after solubilization and subcutaneous injection (group #4), compared to the soluble, doubly-adjuvanted vaccine administered subcutaneously.

### EXAMPLE 4

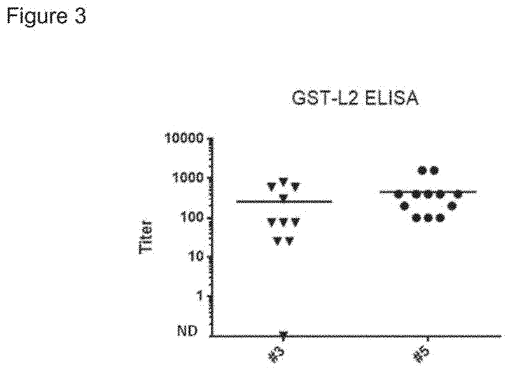

To evaluate the versatility of the above described dry-powder vaccine formulation procedure, an alternative HPV-L2x3 antigen (designated as OVX313-PfTrx-HPV-L2x3) sharing the same immune-epitope, but differing both in size (247560 Da vs. 18294 Da) and total net charge (+6 vs. +2) from the reference PfTrx-HPV16-L2x3 antigen and with a reportedly higher immunogenicity, was subjected to spray-drying and converted into an MPLA-adjuvanted powder form. To test the immune performance of this alternative dry-powder vaccine, 1 mg of OVX313-PfTrx-HPV-L2x3-containing powder (corresponding to approximately 20 μg of protein antigen) were administered intra-tracheally to 13 Balb/c mice following the same immunization schedule described in Example 2 (sec FIG. 2a). After mouse sacrifice and blood collection, the immune-sera were analyzed by GST-L2 capture ELISA and the resulting anti-HPV L2 titers were compared with those obtained with the dry-powder formulated and intra-tracheally administered PfTrx-HPV-L2x3 vaccine. As shown in FIG. 3, a slightly higher anti-HPV L2 antibody titer (average value of 1:500, compared to the average 1:255 titer achieved with PfTrx-HPV-L2x3), was obtained upon pulmonary vaccination with the dry-powder formulated OVX313-PfTrx-HPV-L2x3 vaccine.

### NON-STANDARD LITERATURE CITED

Nardelli-Haefliger et al. (2005), Vaccine, 23:3634-3641
WO 2010/003465
WO 2010/070052
WO 2017/211886

1. A dry pharmaceutical composition for inhalation comprising an antigen and an amphiphilic immune stimulant, wherein said pharmaceutical composition was produced by spray-drying.

2. The dry pharmaceutical composition of claim 1 further comprising at least one bulking agent.

3. The dry pharmaceutical composition of claim 1, wherein said antigen is comprised at a proportion of from 0.1% (w/w) to 10% (w/w) in said dry pharmaceutical composition.

4. The dry pharmaceutical composition of claim 1, wherein said antigen is comprised at a proportion of from 0.3% (w/w) to 2.5% (w/w), preferably of about 2% (w/w) in said dry pharmaceutical composition.

5. The dry pharmaceutical composition of claim 1, wherein said amphiphilic immune stimulant is comprised at a proportion of from 0.1% (w/w) to 10% (w/w) in said dry pharmaceutical composition.

6. The dry pharmaceutical composition of claim 1, wherein said amphiphilic immune stimulant is an agonist of a toll-like receptor (TLR).

7. The dry pharmaceutical composition of claim 1, wherein said amphiphilic immune stimulant is monophosphoryl lipid A.

8. The dry pharmaceutical composition of claim 1, wherein said spray-drying comprises spraying a solution comprising the compounds as specified at elevated temperature into a stream of a gaseous drying agent.

9. The dry pharmaceutical composition of claim 1, wherein said antigen comprises at least one peptide comprising an amino acid sequence corresponding to amino acids 20 to 38 of the HPV16 L2 polypeptide.

10. A dry pharmaceutical composition obtained or obtainable by spray-drying a solution comprising an antigen, an amphiphilic immune stimulant, and, optionally, a bulking agent.

11. (canceled)

12. (canceled)

13. (canceled)

14. A method for manufacturing a dry pharmaceutical composition comprising spray-drying a solution comprising an antigen, an amphiphilic immune stimulant, and, optionally, a bulking agent.

15. A kit comprising the dry pharmaceutical composition according to claim 1 in a housing.

16. The dry pharmaceutical composition of claim 1, wherein said antigen is a thermostable polypeptide, wherein said antigen comprises a thioredoxin, and/or wherein said antigen comprises at least one antigenic epitope of a papillomavirus.

17. The dry pharmaceutical composition of claim 2, wherein the at least one bulking agent is selected from mannitol, lactose, and trehalose.

18. The dry pharmaceutical composition of claim 1, wherein said amphiphilic immune stimulant is an agonist of TLR4.

19. The dry pharmaceutical composition of claim 18, wherein said amphiphilic immune stimulant is selected from the list consisting of monophosphoryl lipid A, synthetic lipid A, lipid A analogs, lipid A mimetics, cytokines, saponins, lipopolysaccharide (LPS) of gram-negative bacteria, and endotoxins.

20. A method of treating and/or preventing an infection in a subject, comprising contacting said subject with a dry pharmaceutical composition according to claim 1, thereby treating and/or preventing an infection.

21. The method of claim 20, wherein said contacting comprises inhalation of said dry pharmaceutical composition.
